(19) United States
(12) Patent Application Publication
(10) Pub. No.: US 2010/0317974 A1
(43) Pub. Date: Dec. 16, 2010

(54) DETECTION OF VULNERABLE PLAQUES BY RAMAN SPECTROSCOPY

(75) Inventors: Robert R. Alfano, Bronx, NY (US); Cheng-Hui Liu, Flushing, NY (US); Wubao Wang, Flushing, NY (US); Vidyasagar Sriramoju, Dobbs Ferry, NY (US)

Correspondence Address: Fish & Richardson P.C. / CUNY;Research Foundation of the City University of New, P.O. Box 1022, Minneapolis, MN, 55440-1022, US

(73) Assignee: Research Foundation of the City University of New York, New York, NY (US)

(21) Appl. No.: 12/684,739

(22) Filed: Jan. 8, 2010

Related U.S. Application Data
(60) Provisional application No. 61/193,931, filed on Jan. 9, 2009.

(51) Int. Cl. A61B 6/00

(52) U.S. Cl. 600/476

(57) ABSTRACT

An apparatus and method of use for detecting vulnerable plaque (VP) in arterial walls is provided. The method includes measuring whether the Raman spectrum of adipose (lipid) tissue signal is present in a Raman signal from aortic intimal wall tissue. The Raman vibration modes for VP are strong bands at about 1435 cm⁻¹, about 2850 cm⁻¹, and about 2892 cm⁻¹ and will be present when the aortic intimal wall tissue is thin.

### CROSS REFERENCE TO RELATED APPLICATIONS

The present application claims the benefit of U.S. Provisional Patent Application Ser. No. 61/193,931, filed on Jan. 9, 2009, the contents of which are hereby incorporated by reference in its entirety.

### BACKGROUND

Atherosclerosis is a diffuse, chronic inflammatory disorder leading to the buildup of fatty deposits on the inside of the artery walls. In the United States alone, more than 1 million people die each year of heart attacks related to coronary artery disease and plaques built up inside the arterial walls. Atherosclerotic plaque builds up quietly, usually causing no symptoms until reaching an advanced stage. Thus, the detection of atherosclerotic plaque is important for the diagnosis, treatment, and prognosis of atherosclerosis and other cardiovascular disease.

Not all plaque presents the same risk for sudden major cardiac events such anunstable angina, myocardial infarction, and sudden cardiac death. It has been found that vulnerable plaque (VP), which is a soft lipid pool covered by a thin fibrous cap, provides an increased risk of thrombosis and rapid stenosis progression. Thus, the detection of VP can be particularly important for identifying and treating patients at risk as well as monitoring disease progression. The types of VP most prone to rupture are inflamed thin-cap fibroatheroma (TCFA). The major components of TCFA are a lipid-rich atheromatous core, a thin fibrous cap, and expansive remodeling. These plaques often have a thin fibrous cap, which is generally less than 100 μm or less than 65 μm, and are a more specific precursor of plaque rupture due to tissue stress.

The traditional clinical tools for detecting plaque, such as intravascular ultrasound, optical coherence tomography, and high-resolution magnetic resonance imaging are limited by their poor sensitivity and prediction of rupture of VP, and give little or no information regarding molecular and cellular mechanisms. A key limitation has been the lack of available techniques with an appropriate indicator for probing the rupture of vulnerable atherosclerotic plaque in vivo. Thus, an apparatus and a method of detecting VP and/or monitoring the degree of VP at the aorta intimal surface and optionally treating VP are needed. Preferably, the method should be simple, inexpensive, and accurate.

### SUMMARY OF THE INVENTION

The present invention provides a method for detecting vulnerable plaque, a method for treating a patient, and an apparatus useful for detecting VP in a patient. It has been found that aortic fatty tissue and human calcified atherosclerotic tissue has characteristic Stokes Raman vibration bands of 1435 cm⁻¹, 2850 cm⁻¹ and/or 2892 cm⁻¹ where the presence of these bands indicates the presence of VP. These three Raman modes, which are the main lipid C—H vibration bands, have sharp spectrum, strong features and high stability with varied environments including temperature, and can be used as a fingerprint of VP.

Thus, a method of detecting vulnerable plaque is provided which comprises: a) irradiating a cardiovascular tissue sample with monochromatic light, b) detecting Raman signal scattering from the cardiovascular tissue sample at one or more of about 1435 cm⁻¹, about 2850 cm⁻¹, and about 2892 cm⁻¹, and at one or more background frequency, and c) processing the Raman signal to obtain spectroscopic information at one or more of about 1435 cm⁻¹, about 2850 cm⁻¹, or about 2892 cm⁻¹, wherein the spectroscopic information at one or more of about 1435 cm⁻¹, about 2850 cm⁻¹, or about 2892 cm⁻¹ indicates the presence or absence of vulnerable plaque in the cardiovascular tissue sample.

Also provided herewith is a method of treating a patient comprising detecting vulnerable plaque as defined above and targeting said vulnerable plaque for treatment.

The present invention also provides an apparatus for detecting vulnerable plaque in a sample comprising: a source of monochromatic light, a photodetector, a probe comprising a fiber optic endoscope, and a processor configured to provide spectroscopic information at one or more of about 1435 cm⁻¹, about 2850 cm⁻¹, or about 2892 cm⁻¹, calculate the thickness of sample intimal wall, and indicate the presence or absence of vulnerable plaque in the sample, where the fiber optic transmits light from the monochromatic light source to a sample and scattered light from a sample to the photodetector. The processor processes data obtained from the photodetector.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

FIG. 1a is a Raman spectrum of Aorta intimal wall tissue, fat from aorta adventitial wall, and cholesterol (powder, Sigma Corp.). Exposure time was 5 seconds. Excitation wavelength at 633 nm, Scan Center at 680 nm (300 cm⁻¹ to 1800 cm⁻¹) and 760 nm (2000 cm⁻¹ to 3200 cm⁻¹).

FIG. 1b is a Raman spectra of Aorta intimal wall tissue normalized to baseline at 1341 cm⁻¹; fat tissue is from aorta adventitial wall, and cholesterol (powder, Sigma Corp.). Exposure time was 5 seconds. Excitation wavelength at 633 nm, Scan Center at 680 nm.

FIG. 1c is a Raman spectrum of Aorta intimal wall tissue, normalized to baseline at 2770 cm⁻¹, fat tissue is from aorta adventitial wall, and cholesterol (powder, Sigma Corp.). Exposure time was 5 seconds. Excitation wavelength at 633 nm, Scan Center at 760 nm.

FIG. 2a is a Raman spectrum showing the Intensity changes in the region from 1250 cm⁻¹ to 1700 cm⁻¹. Scan center at 680 nm, (mode of 1435 cm⁻¹). Spectra are shown for various thicknesses (100 μm, 400 μm, and 1400 μm) of intimal layers on the top of fat tissue.

FIG. 2b is Raman spectra showing the Intensity changes in the region from 2500 cm⁻¹ to 3200 cm⁻¹. Scan center 760 nm, modes of 2850 cm⁻¹ and 2892 cm⁻¹. Spectra are shown for various thicknesses (100 μm, 400 μm, and 1400 μm) of intimal layers on the top of fat tissue.

FIG. 3a is a graph showing the peak intensities of the Raman spectral mode of 1435 cm⁻¹ of aorta fat as a function of the thickness of aorta intimal wall tissue layers lying on the top of the fat (filled squares). The standard deviation error bars were obtained from the statistic analysis over twelve measurements. The solid curve is a fit to the data using an exponential decay.

FIG. 3b is a graph showing the peak intensities of Raman spectral modes of 2850 cm⁻¹ and 2892 cm⁻¹ of aorta fat versus the thickness of aorta intimal wall tissue layers lying on the top of the fat tissue. The standard deviation error bars were obtained from the statistic analysis over twelve measurements. The solid curve is a fit to the data an exponential decay.

FIG. 4a is a schematic of an artery having thick and thin VP with five measurement sites identified and used for ratio meter detecting processing for calcified plaque and VP.

FIG. 4b shows five Raman spectra taken at the five locations shown in FIG. 4a. This ratio meter scan processing with a catheter head probe provides the raw Raman spectra of aorta intimal wall, where site (1) is a pure intima only site, site (2) is a thick VP site, site (3) is a pure intima only site, site (4) is a thin VP site, where the signal is weaker than for site (2), and site (5) is intima only.

FIG. 4c shows five Raman spectra taken at the five locations shown in FIG. 4a. This ratio meter final scan processing with a catheter head probe provides the background corrected Raman spectra of aorta intimal wall, where site (1) is a pure intima only site, site (2) is a thick VP site, site (3) is a pure intima only site, site (4) is a thin VP site, where the signal is weaker than for site (2), and site (5) is intima only.

FIG. 5 is a diagram of a Raman ratio meter for detecting VP having two fibers.

FIG. 6 is a diagram of a fiber-optic Raman micro imaging endoscope.

### DETAILED DESCRIPTION OF THE PREFERRED EMBODIMENTS

This invention relates generally to an apparatus and methods of detecting and optionally treating vulnerable plaques (VP) in arterial walls by measuring whether the Raman spectrum of adipose (lipid) tissue signal is present in the Raman signal of aortic intimal wall tissue. Thin cap layers overcoating a lipid adipose layer region can be detected by measuring the Raman spectra and the changing intensities of particular Raman modes. This spectroscopic information reveals the thickness of intimal arterial wall, plaque size and components, including the fatty core and lipid pool. If Raman signals from the adipose tissue are observed, the tissue cap layer is too thin and the VP is present. An optical fiber probe such as a catheter head probe can be used within the artery to detect the Raman signal.

Three Raman modes at 1435 cm⁻¹, 2850 cm⁻¹ and 2892 cm⁻¹ are new molecular spectroscopic fingerprint indicators to determine the presence of VP in a cardiovascular tissue sample. The in situ monitoring of the development of fatty-streaks and lipid core in aorta walls and determination of the thickness change of aorta intimal wall at different stages of atherogenesis to find regions of VP is also provided. Raman signal observed from lipids indicates the artery tissue is only a thin wall of tissue, or cap, and VP is present. The Raman vibration modes from the lipids for determining the presence of VP include the strong bands at 1435 cm⁻¹, 2850 cm⁻¹ and 2892 cm⁻¹. These modes reveal the strong vibration strength of the C—H stretching vibration region of lipid for fat/lipids under a thin tissue layer. The overlying tissue layer does not have Raman bands in these regions but acts to attenuate the intensity of the Raman signal from the underlying lipid layer. Thus, the intensity attenuation of these Raman peaks is a function of thickness of aortal intimal wall tissue layers on the top of the fat tissue. Thus, one or more of the Raman vibrational bands at 1435 cm⁻¹, 2850 cm⁻¹ and 2892 cm⁻¹ are used as new indicators to determine the presence of VP and monitor the changes of arthrosclerosis lesions VP and stages of the plaque formation, cumulating in ruptured plaque in arteries. The Raman spectral data may also be used to monitor the effects of diet and lipid-lowering therapy on atherosclerotic plaque development in vivo by combining this Raman technology with a method of calculating atherosclerotic plaque using Raman spectroscopy to obtain data on both types of lesions.

The intensity or ratio of these three Raman vibrational modes are indicators useful for the analysis of the changing composition of the fatty-streak and lipid core concentrations in aorta walls, capped by tissue which may vary in thickness. Both the intensity and the ratio, independently or together can be used to monitor the thickness changes of aorta intimal wall at different types of atherosclerosis and determine the presence of VP.

While the invention preferably uses the lipid bands at 1435 cm⁻¹, 2850 cm⁻¹ and 2892 cm⁻¹, any Raman signal from the lipid may be used to determine the presence of VP. Thus, Raman signal from any lipid, include apolipoproteins, saturated lipids, (poly-) unsaturated lipids, triglycerides, trans-fatty acids, and cholesterol may be used. This signal may be at one of the three main bands as listed above, or it may be at any other location in the Raman spectra for the lipid, such as bands at 1293 cm⁻¹ and 1641 cm⁻¹. However, since the main characteristic fingerprint Raman vibration modes of adipose tissue at 1435 cm⁻¹, 2850 cm⁻⁻¹ and 2892 cm⁻¹ have an intensity approximately four times stronger than that of the other modes, it is preferred to use at least one of the three main modes.

In some embodiments, it is preferred to use at least two of the adipose tissue modes at 1435 cm⁻¹, 2850 cm⁻¹ and 2892 cm⁻¹. In other embodiments, it is preferred to use all three of the adipose tissue modes at 1435 cm⁻¹, 2850 cm⁻¹ and 2892 cm⁻¹. In other embodiments, it is preferred to use the two adipose tissue modes at 2850 cm⁻¹ and 2892 cm⁻¹. In yet other embodiments, it is preferred to use the adipose tissue modes at 1435 cm⁻¹, 2850 cm⁻¹ and 2892 cm⁻¹ as well as additional modes, such as the modes at 1293 cm⁻¹ and 1641 cm⁻¹.

Using the lipid Raman signal, the location of the VP regions as well as the thickness of the VP in the aortal tissue can be measured and documented. The intensities of characteristic Raman spectral modes of the aorta fat are attenuated when the cap layer thicknesses of aortic intimal wall increases. Thus, a map of the thickness of intimal wall as the wall thickness changes due to variations of lesions and type of atherosclerosis can be obtained by measuring Raman signal of the tissue as the probe moves through along the intimal wall. This map can be used for the diagnosis or prognosis of atherosclerotic disease state. For example, the map can be used to determine whether a patient is a vulnerable patient. It can also be used to determine the effectiveness of diet or of a treatment regime.

An exponential decay curve may be used to display the attenuation process, and this decay curve indicates the changing thickness of lumen intimal tissue and indicates the presence or absence of vulnerable plaque. The exponential decay curve may be mapped over the length of the intimal wall as measured by, for example, a catheter head probe. This decay curve can aid in the diagnosis or prognosis of atherosclerotic disease state by providing information on the presence of VP as well as the location, amount, and cap thickness.

A Raman ratio meter may be used to measure Raman intensity at two or more Raman frequencies to get a measurement of the lipid/tissue cap region. The Raman signal from the lipid adipose layer can generally be detected if the cap covering the layer is thin, (i.e., from 5 μm to 200 μm). Thus, when Raman scatter from the lipid adipose layer is observed, the tissue cap layer is too thin and the sample is VP.

This method can be used inside an artery of a patient (e.g., a human) in situ. In one embodiment, the method is particularly used in one or more portions of the large arteries closest to the skin, such as the carotid or femoral arteries. In another embodiment, the method is used in the coronary arteries, the small arteries close to the heart, since these arteries commonly have the most ruptures. Additionally, the presence of fatty tissue in other vessels and organs may also be determined using the methods and systems of the present invention.

In some embodiments, both VP and calcified plaque are detected. As disclosed in U.S. Pat. No. 5,293,872, hereby incorporated by reference in its entirety, the 957 cm-1 Raman mode can be used to detect calcified plaque. Thus, by detecting one or more of the Raman modes at 1435 cm⁻¹, 2850 cm⁻¹ and/or 2892 cm⁻¹ in combination with the 957 cm⁻¹ Raman mode, both VP and calcified plaque are detected. This can allow further diagnosis and/or prognosis of a patient. Thus, the present invention allows for distinguishing between calcified atherosclerotic tissue and normal cardiovascular tissue as well as for determining the presence of VP.

The Raman signal can be detected using a fiber optic probe-based endoscopic system. Optionally, an imaging system is also used to image the cardiovascular tissue as the Raman signal is obtained at the same or proximate location in the intimal wall.

The Raman spectra and the changes of intensities of Raman modes reveal the thickness of intimal arterial wall, plaque size and components, fatty core, lipid pool and its developments. If Raman signal from fat is observed the tissue cap layer is too thin and the sample is designated a VP.

The thin cap overcoating the lipid layer in VP that attenuates the Raman signal of the lipid layer is generally between 5 and 200 μm. Within this range, the Raman signal from the lipid adipose layer can be detected as described herein. Thus, when Raman scatter from the lipid adipose layer is observed, the tissue cap layer is too thin (e.g., less than 65 μm or less than 100 μm) and the region is defined as VP. For thicker caps, the lipid adipose layer is not detected and the lipid adipose is not considered VP. A minimum intensity for the Raman bands at 1435 cm⁻¹, 2850 cm⁻¹ and/or 2892 cm⁻¹ may be set as a decision point such that signal from the adipose lipid layer. While the lipid signal may be observed from a tissue sample where the cap is thick (i.e., over 100 μm), a decision point may be set to define this signal as not VP. Similarly, a decision point may be set at, for example when the signal is obtained but the cap layer has a thickness of 75 μm, 75 μm, 100 μm, 125 μm, 150 μm, 175 μm, 200 μm, 250 μm, 300 μm, 400 μm, 500 μm, 600 μm, 700 μm, 800 μm or more. This signal can be defined as for thick cap where the underlying lipid adipose layer is not VP. Additionally, while the cap is generally at least 5 μm thick, lipid adipose layers under caps thinner than 5 μm can be detected as well. Smaller thickness about 1 μm, 2 μm, 3 μm, or 4 μm of tissue of layer of artery can be probed to detect the VP regions. When a weak signal is obtained from a lipid pool where the lipid pool itself is thin, the signal may be defined as either VP or not VP, depending on the parameters set for the processor and the intensity of the spectral information.

For aorta or sections thereof having a thick tissue layer, the excitation and emission signals at the three Raman lipid modes are absorbed by the tissue and the lipid bands are not seen. Thus, when no Raman signal is present, there is no VP.

Some embodiments include a method for detecting VPs using Raman spectroscopy by obtaining spectroscopic information at 1435 cm⁻¹, 2850 cm⁻¹, and/or 2892 cm⁻¹, which are characteristic scattering bands of aortic fatty tissue. When Raman signal is observed at these wavenumbers, the tissue cap layer is too thin and the sample is VP.

Another embodiment is an apparatus that includes an excitation source and a photodetector for measuring Raman scatter in a cardiovascular tissue sample. The apparatus also includes a processor, which uses the Raman spectroscopic information to determine whether the cardiovascular tissue sample contains at least one region of VP.

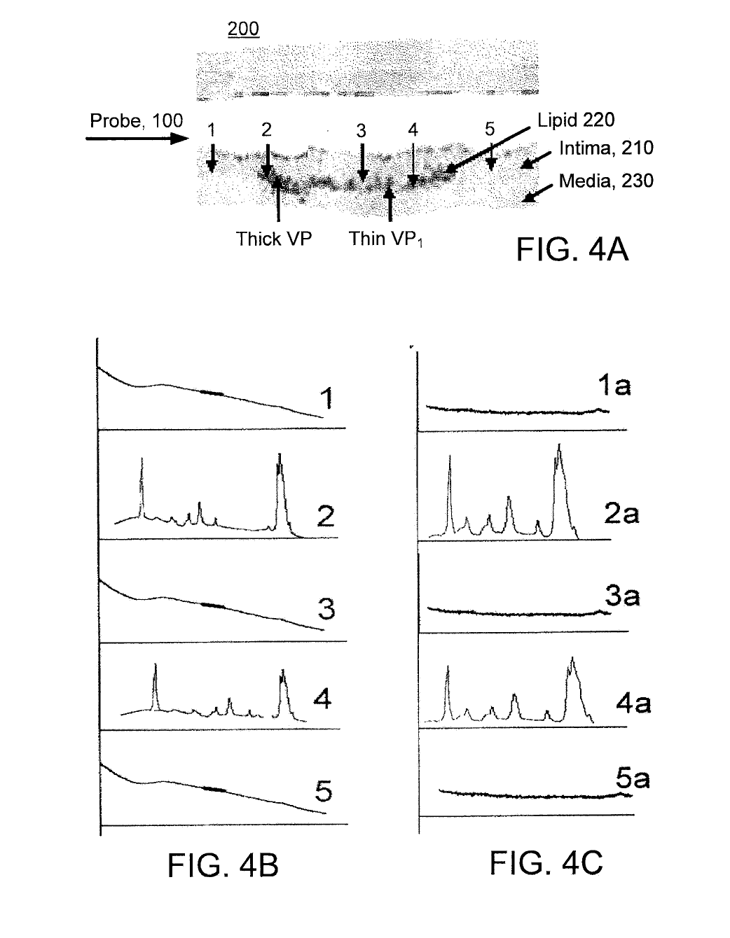

FIG. 4 shows a Raman Ratio Meter useful for detecting and processing calcified plaque and VP signals in regions of an artery. A catheter head probe (100) is inserted into the artery (200) which contains intima (210) as well as pockets of lipid (220). The intima layer (210) has been placed on a media layer (230). In this example, it is inserted from the left and moves to the right, obtaining data along the artery at positions 1-5. As the Raman Ratio meter scans though the artery (200) with a catheter head probe (100), Raman spectra are obtained, as shown in FIG. 4b at each of the sites and the data is processed. Signal is obtained for each of the aorta intimal wall at site 1, showing no Raman bands but having a sloping background. The raw signal at site 2 shows signal from a thick layer of VP with a thin layer of tissue overlaying the adipose lipid layer. Site 3 again shows the signal when the probe is over pure intima. Site 4 shows the signal from a thin VP site and a thin overlying tissue layer. The signal here is weaker than at site 2. Site 5 shows a scan of the intima wall again, where there is no VP. The signal at sites 1-5 are processed by subtracting the background spectra from the tissue samples at sites 1-5 and the results are shown in FIG. 4c.

Spectroscopic information, including the raw data, background subtracted date, optionally other spectroscopic information, and calculated results such as an exponential decay of the attenuation process can be displayed on a display device, such as a monitor. The display may occur in real time and can show the area of the fatty-streak or/lipid core under the intimal wall as determined by calculating changes in the thickness of the intimal wall. Optionally, the areas of ruptured plaques may be displayed as well.

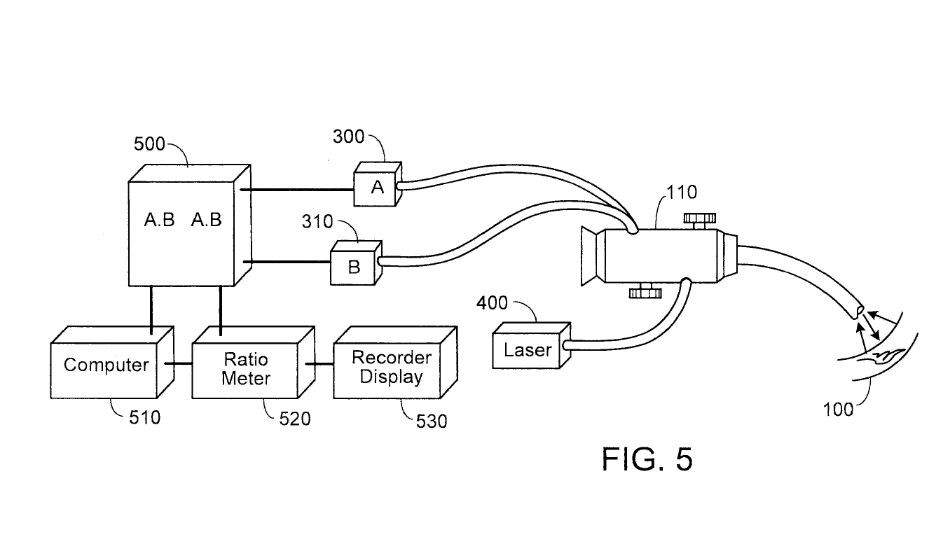

FIG. 5 illustrates one embodiment of the invention where signal is obtained and processed through a Raman ratio meter for VP. A filter probe (100) enters into an artery, Raman Signal A is from arterial tissue, Raman signal B is from calcified plaque or VP. Signal from fibers A and B pass through the appropriate notch filters and then through narrow band filter selected to correspond to the Raman peaks at 957 cm⁻¹, 1293 cm⁻¹, 1435 cm⁻¹, 1647 cm⁻¹, 2850 cm⁻¹ and 2892 cm⁻¹ (110). The signal is then sent to photodetectors (300) and (310). A laser (400) having a narrow band filter is used as the excitation source. After exiting the photodetector (300), signal is sent to an electronic converter (500) which is attached to a computer (510), a ratio meter (520). This converter (500) obtains spectroscopic information which is a ratio is equal Raman peak intensity I_{B} to background intensity I_{A} (I_{B}/I_{A}) on computer screen (530) which then shows an intensity ratio, Raman spectrum, or both.

In some embodiments, a Raman spectral difference meter is used instead of or in addition to the Raman ratio meter. In some embodiments, additional fibers are used for additional Raman signal, imaging, or excitation.

In some embodiments a ratio meter is used where the ratio meter (520) comprises a narrow band filter-semiconductor diode laser, a narrow band gap filter at the laser line, and two or more optical fibers filtered at two or more wavelengths in the lipid and tissue spectral regions, and two or more photomultipliers. Computer software is used to obtain the ratio of one or more peaks of Raman frequencies to determine VP using one or more shift of about 1435 cm⁻¹, 2850 cm⁻¹, or 2892 cm⁻¹ and optionally in the calcified plaque region (Raman shift at 957 cm⁻¹). From the measured ratio, the present of VP and/or calcified regions is determined.

In some embodiments a spectral difference meter is used where the difference meter (520) comprises a narrow band filter-semiconductor diode laser, a narrow band gap filter at the laser line, and two or more optical fibers filtered at two or more wavelengths in the lipid and tissue spectral regions, and two or more photomultipliers. Computer software is used to obtain the difference between one or more peaks of Raman frequencies to determine VP using one or more shift of about 1435 cm⁻¹, 2850 cm⁻¹, or 2892 cm⁻¹ and optionally in the calcified plaque region (Raman shift at 957 cm⁻¹). From the measured ratio, the present of VP and/or calcified regions is determined.

In some embodiments, an imaging endoscope system used in the present invention. The endoscope system comprises a single mode optical fiber delivering both the excitation beam and the Raman scattered signal light. Several optical filters are used to filter the signal. The imaging endoscope system also contains a balloon or umbrella-shaped end unit attached at the distal end and side of the fiber optic probe. A ball lens is used to couple the optical fibers. The probe used in this embodiment has a high spatial resolution. The measured spectrum shows a depth-resolution of 5 μm to 1.2 mm for the detection of adipose lipid signal under aorta intima tissue. In some embodiments, the measured spectrum shows a depth resolution of at least 10 μm. In some embodiments, the depth resolution is at least 100 μm.

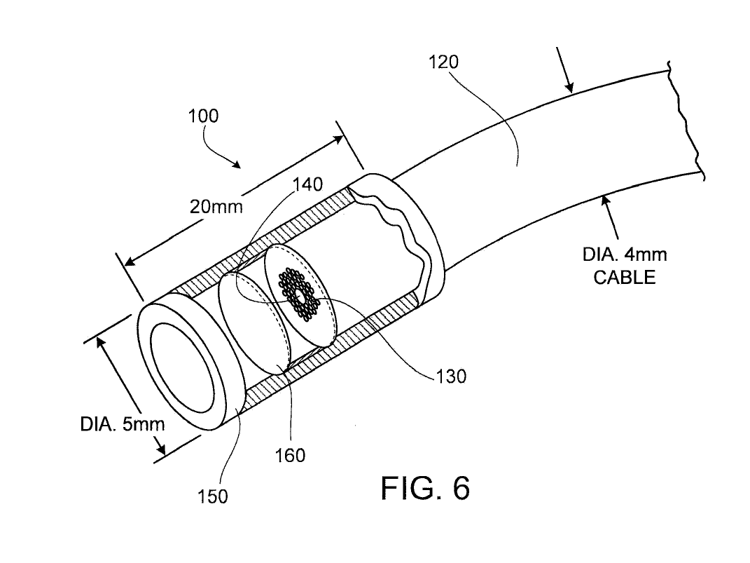

In some embodiments a fiber-optic Raman micro imaging endoscope system as described in U.S. Pat. No. 5,293,872 and shown in FIG. 6 is used for detection of VP and optionally calcified plaque region of arteries. This optical fiber bundle assembly (100) includes a cable (120) having an outer diameter of about 4 mm and houses a number of optical fibers (130) which are preferably made of quartz, sapphire or any other infrared-transmitting material. An excitation fiber (140) is centrally disposed in the cable has a diameter of about 400 um and conveys a beam of visible or infrared monochromatic light to the tissue being tested. Additional optical fibers (130), each having a diameter of about 100 to 200 um, surround the excitation fiber and convey the Raman scattered light from the tissue being tested to the interferometer. The optical fibers (130), taken together, have a diameter of about 2.5 to 4.0 mm. The fibers (130) are surrounded by a housing (150) that is attached to the cable. This housing is about 20 mm in length and about 5 mm in diameter and is preferably made of metal. If desired, a focusing lens (160) for focusing the light entering and leaving the optical fibers may be mounted within the housing. The lens (160), which is preferably made of quartz or sapphire, is preferably 3 mm in diameter and has a focal length of 7 mm.

In some embodiments, the micro-Raman imaging apparatus is a Raman spectroscopic system including a monochromatic light source, optical collection of the backscattered signal with a spectrograph, a CCD detector, and a micro Raman imaging endoscope system. In this embodiment, the micro Raman endoscope has a single mode fiber, a probe with multiple heads, side prisms, lenses, four-90° umbrellas for screening blood or intervening fluid and cellular plasma, and two arms fiber probes for calibration (i.e., for the fat and a mixture of lipid and tissue).

In some embodiments, the probe contains at least two channels. One of the probe channels is designed to collect calibration signals and one of the probe channels is used for both Raman excitation and emission signals at the sampling location(s). Computer software can perform real-time conversion of the Raman and calibration signal to provide real-time signal and determination of the adipose layer and the thickness of an overlying cap. This signal can then be displayed. In some embodiments, the probe contains four channels as particularly described and disclosed as FIG. 10 of U.S. Pat. No. 5,293,872.

In some embodiments, the endoscope includes an outer cuff that is sized and shaped to fit within an artery or other blood vessel. The cuff surrounds all but the tip of a cable having several channels. An optical fiber bundle, which is used to illuminate the interior of the artery for imaging on a monitor, is mounted within a first channel. A fiber optic bundle is mounted within a second channel and is used to convey the monochromatic light used as the Raman excitation source to the arterial tissue and to collect the resulting Raman scattered light. Additionally, the fiber optic bundle can be used for conveying fluorescence excitation and emission for the detection of fibrous atherosclerotic tissue. Additionally the fiber optic bundle can be used for conveying an additional laser line which is at a wavelength in resonance with one of 1435 cm⁻¹, 2850 cm⁻¹, or 2892 cm⁻¹. An additional optical fiber may be mounted within a third channel. This fiber can transmit powerful laser light to any detected VP or atherosclerotic tissue for ablation. A length of tubing for use in aspirating ablated tissue and other debris from the artery may be mounted within a fourth channel.

Monochromatic light, as used herein, refers to light having a single wavelength as produced by a laser or other monochromatic light source. The monochromatic light is preferably in the visible or near IR region. In some embodiments, the source of monochromatic light is selected from an ion laser (Ar⁺, Kr⁺, He—Ne), a solid-state laser (YAG, Ti:sapphire, Forsterite, Cunyite, etc), a diode laser (GaAs), a dye laser, LIGO, or LISO. In some embodiments, the monochromatic light is from a laser source having an excitation source at, for example, 633 nm, 785 nm, 800 nm, or 632 nm.

In some embodiments, a second wavelength of monochromatic light may be used. Preferably, this wavelength is at or near the Stokes shift of one of the adipose lipid bands (i.e., at 1435 cm⁻¹, 2850 cm⁻¹, or 2892 cm⁻¹). In this embodiment, a resonance effect is used, where the first light source having a first wavelength of monochromatic light excites the fat molecules and causes emission at the fat band. The second light source having a second wavelength of monochromatic light then creates a resonance with the Raman signal at an adipose lipid band when the intimal layer is thin and the fat signal is detected. This embodiment is particularly useful when it is important to detect VP having a thicker intimal wall than can be easily measured without using a resonance effect.

The scattered Raman signal is detected using a photodetector. Optionally, the signal is collected with a spectrograph with or without additional filters. In some embodiments, the photodetector is a CCD camera where the signal is first filtered with a notch filter at laser excitation frequency. Additionally, holographic narrow band filters may be used to remove unwanted scatter. In another embodiment, the photodetector is two or more photomultipliers coupled with two or more narrow band filters, or more particularly holographic narrow band filters.

Based on the intensity of the fat signal, the tissue cap layer thickness may be calculated and optionally fit to an exponential decay model. Thus, the presence of Raman scatter at one or more of about 1435 cm⁻¹, 2850 cm⁻¹, or 2892 cm⁻¹ indicates the presence of VP. The signal may be processed in any one of a number of ways. For example, the signal may be background subtracted using a single wavelength indicative of the tissue but not fat signal, the signal may be background subtracted by subtracting the Raman spectrum of aorta intimal wall or an artery without VP. The spectroscopic information may include the maximum signal intensity or, alternatively, another parameter may be used such as, for example, the full-width-half-max, or a fitted peak area. Preferably, when a ratio is calculated, both the signal from the adipose lipid layer and the background signal are processed the same way.

Attenuation due to tissue cap layer thickness can be distinguished from the thickness of the VP layer by calculating a ratio or subtracting background signal at, for example 1350 cm⁻¹ (for the 1435 band) or 2750 cm⁻¹ (for the 2850 cm¹, or 2892 cm⁻¹ bands).

A ratio calculated from the Raman signal measured at two Raman lines (i.e., at the lipid frequency and at one or more frequencies where the lipid is not present) can be used to determine the presence of VP without the need for measuring the full Raman spectrum. This allows for faster measurement and analysis. It also allows for the use of a simpler Raman probe (i.e., a system having filters and two PMT may be used instead of a monochrometer and CCD system).

When calcified plaque is detected by measuring a signal at about 957 cm⁻¹, this signal may be ratioed with the signal from the VP. Alternatively, this signal may be ratioed with a background signal.

In some embodiments, the spectral resolution of the apparatus or method is approximately 4 cm⁻¹. Thus, detection and analysis of the Raman bands stated to have a shift of about 1435 cm⁻¹, 2850 cm⁻¹, and 2892 cm⁻¹ are measured with error as: 1435 cm⁻¹±4 cm⁻¹, 2850 cm⁻¹±4 cm⁻¹, and 2892 cm⁻¹±4 cm⁻¹. In other embodiments, the spectral resolution of the apparatus or method is approximately 5 cm⁻¹. In other embodiments, the spectral resolution of the apparatus or method is approximately 3 cm⁻¹. In other embodiments, the spectral resolution of the apparatus or method is approximately 2 cm⁻¹.

The methods and apparatus as described herein may be performed in vivo. A probe may be inserted into a patient's artery or at another location to determine the presence of VP. In certain embodiments, the patient is a mammal. In other embodiments, the patient is a human.

The methods and apparatus may be used to determine whether a patient has VP. The method as described herein may also be used to track or verify the efficacy of medical treatment or diet on the progression, stability or potential regression of plaque within a patient over time. It may also be used to monitor the change of arthrosclerosis lesions, or monitor the stage of stages of plaque rupture.

Some embodiments of the present invention comprise the identification of a vulnerable patient. Vulnerable plaque is detected in the cardiovascular tissue of the patient by obtaining spectroscopic information at one or more of 1435 cm⁻¹, 2850 cm⁻¹, or 2892 cm⁻¹. This information, which includes information as to the thickness of the tissue cap and the depth of the underlying adipose lipid, can be used to quantify the patient as a vulnerable patient when the amount of vulnerable plaque is over a threshold amount. The tissue cap thickness and depth of underlying adipose lipid define the vulnerable plaque, the amount of each of these throughout the measured cardiovascular tissue and the specific locations within the cardiovascular system define when the patient is a vulnerable patent.

In one embodiment of the present invention, a patient is treated after determining that VP is present, unstable, progressing (for example, the patient may be suffering from atherosclerotic vascular disease or suspected of being at risk of experiencing a plaque rupture and/or an occlusive thrombotic event). The method comprises detecting VP and targeting the VP for treatment. Treatment is administered to at least one of the targeted plaques.

Such treatment may occur at the same time as the detection of the VP and optionally calcified plaque. Since the measurement of the Raman signal is a real time event, the detection and determination of the requirement for treatment can occur in real time. Thus, a patient may be treated during the same event as the VP detected. For example, the fiber probe used to detect the Raman signal may contain the means to treat the VP and/or calcified plaque. Alternatively, the treatment may occur at a later time.

Treatment of the VP can include any suitable treatment method. Suitable treatments can then be administered, for example, balloon angioplasty, laser angioplasty, heated balloon (RF, ultrasound or laser) angioplasty, surgical atherectomy, laser atherectomy, the placement of an appropriate stent, a pharmacological treatment such as the administration of anti-coagulants, fibrinolytic, thrombolytic, anti-inflammatory, anti-proliferative, immunosuppressant, collagen-inhibiting, or endothelial cell growth-promoting agents. Any other conventional local or systemic treatments effective for reducing or eliminating inflamed plaque may also be used.

Optionally, additional measurements of the VP and optionally calcified plaque in a patient may be performed during and/or after treatment in order to determine the effectiveness and progression of the treatment.

As used herein, the terms “plaque” and “atherosclerotic tissue” are used interchangeably and refer to the adipose lipid tissue found at the arterial wall.

As used herein, the term “spectroscopic information” includes, for example, peak Raman intensities at various frequencies, integrated peak intensities at various frequencies, calculated area of Raman peaks at various wavelengths, etc. Spectroscopic information also includes the normalized peak Raman intensity, a ratio of peak Raman intensities, a ratio of integrated peak intensities, and a ratio of calculated peak areas.

As used herein, the term “background wavenumber” is a wavenumber of a Raman shift which is not associated with the adipose tissue at 1435 cm⁻¹, 2850 cm⁻¹, 2892 cm⁻¹. The background wavenumber is also not located at 957 cm⁻¹, 1291 cm⁻¹ or 1641 cm⁻¹, or at any other lipid band. The background may also be an average of several different background wavenumbers.

The term “frequency” may also be used to describe the Raman shift at a particular wavenumber, where the shift in frequency from the monochromatic excitation source is the Stokes shift having a particular wavenumber.

A vulnerable patient, as described herein, is a patient who has a high probability of dying of a heart attack within 12 months. While not all vulnerable patients will have VP, patients with a large amount (i.e., over a minimal threshold) of VP are vulnerable patients.

The term “about” or “approximately” means within an acceptable error range for the particular value as determined by one of ordinary skill in the art, which will depend in part on how the value is measured or determined—e.g., the limitations of the measurement system, or the degree of precision required for a particular purpose. Alternatively, “about” can mean a range of up to 25%, preferably up to 15%, more preferably up to 10%, more preferably up to 5%, and more preferably still up to 1% of a given value. Where particular values are described in the application and claims, unless otherwise stated, the term “about” meaning within an acceptable error range for the particular value should be assumed.

As used herein and in the appended claims, the singular forms “a,” “an,” and “the,” include plural referents unless the context clearly indicates otherwise. Thus, for example, reference to “a molecule” includes one or more of such molecules, “a laser” includes one or more of such different lasers having different wavelengths of excitation and reference to “the method” includes reference to equivalent steps and methods known to those of ordinary skill in the art that could be modified or substituted for the methods described herein.

All U.S. patents and published applications cited herein are hereby incorporated by reference.

The above specification, examples and data provide a description of the manufacture and use of the composition of the invention. Since many embodiments of the invention can be made without departing from the spirit and scope of the invention, the invention also resides in the claims hereinafter appended.

### EXAMPLES

The following examples are included to demonstrate preferred embodiments of the invention. It should be appreciated by those of skill in the art that the techniques disclosed in the example, which follows, represent techniques found to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The ability of the present method to detect the presence of VPs in fresh porcine aortic adipose tissue was measured. An excitation wavelength of 633 nm was used. A lipid/tissue structure was built to simulate VP region. The adipose tissue was taken from adventitial fat grown on aorta walls of artery. The thicknesses of porcine aorta wall tissue were obtained by slicing an artery or cutting artery or cut into slices with a thickness of 25 μm-50 μm from an intimal surface. The structure was prepared by placing the various samples of aorta intimal wall tissue layers on top of adipose tissue to vary the total thickness for layered from 50 μm to 2000 μm. The Raman spectra of adipose tissue and tissue were measured and the change in intensities of the Raman modes versus thickness of the aorta intimal wall tissue layers was measured. The total thickness of the aorta intimal wall tissue layers was varied in the range of 50 μm to 1800 μm.

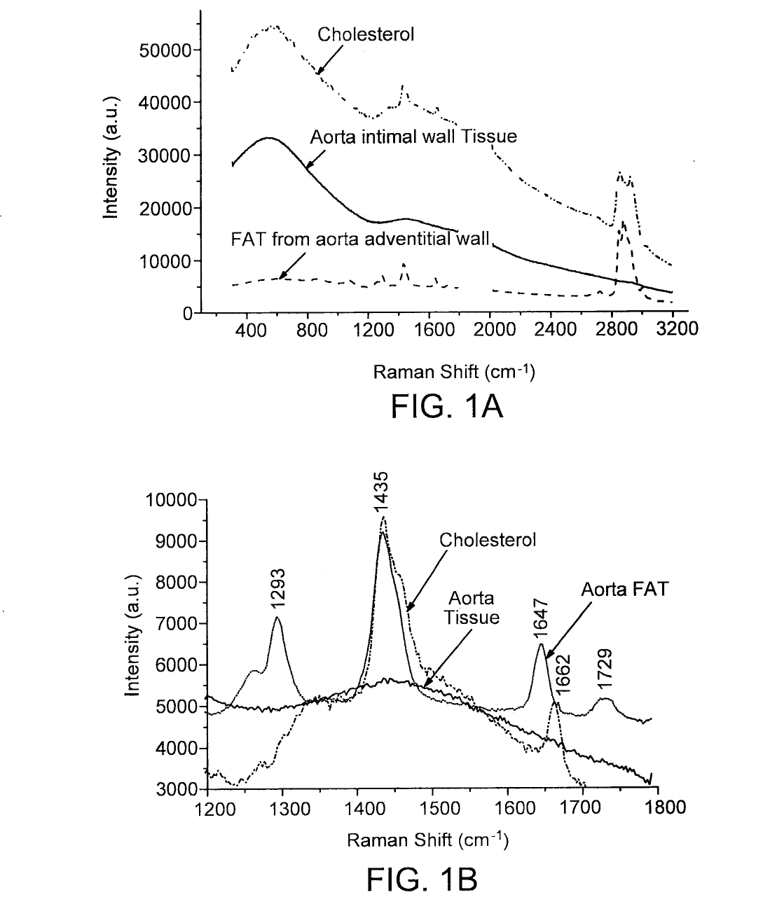

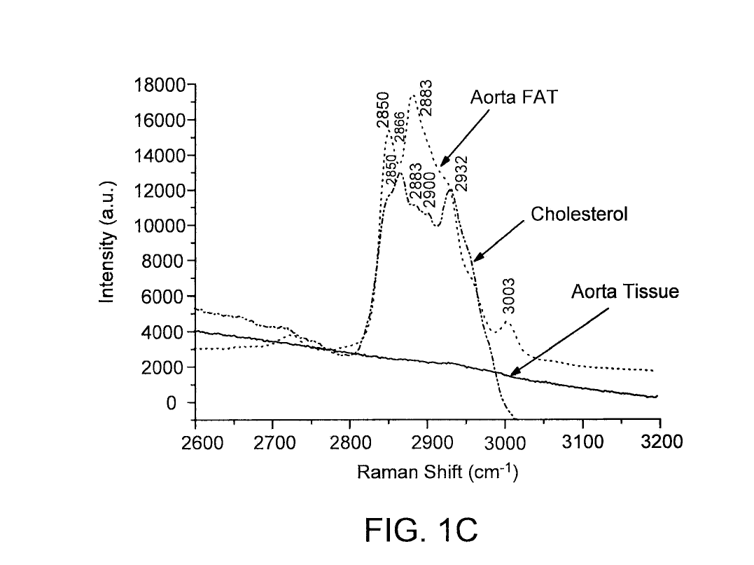

The Stokes Raman vibration modes at 1435 cm⁻¹, 2850 cm⁻¹ and 2892 cm⁻¹ were measured and investigated for pig aorta fatty tissue, aortal intimal wall tissue, chicken fat, laser melted chicken fat oil and cholesterol powder. Raman spectra of aortal fatty, intimal wall tissue and cholesterol powder were plotted in FIGS. 1a, 1b, and 1c. The two Raman spectral scan regions measured were 300 cm⁻¹ to 1800 cm⁻¹ and 2000 cm⁻¹ to 3200 cm⁻¹. The exposure time was 5 seconds. As shown FIG. 1, there are less Raman lines from tissue and more of lines for fat in the regions from 1200 cm⁻¹ to 1700 cm⁻¹ and 2800 cm⁻¹ to 3000 cm⁻¹. Signal from water was not observed. The modes at 1283, 1435, and 1647 cm⁻¹ and associate with lipids/fat (FIG. 1b) and 2850 to 3000 cm⁻¹ (FIG. 1c) assumed with lipids/fat. FIG. 1a shows the original data collected for fatty from aorta adventitial wall and aorta intimal wall which were hold on the surface of quartz slide and cholesterol powder which was held in a quartz cuvette. FIG. 1b shows in spectral scan region of 300 cm⁻¹ to 1800 cm⁻¹ with normalized baseline at 1341 cm⁻¹; and FIG. 1c shows scan region 2000 cm⁻¹ to 3200 cm⁻¹ with normalized baseline at 2771 cm⁻¹.

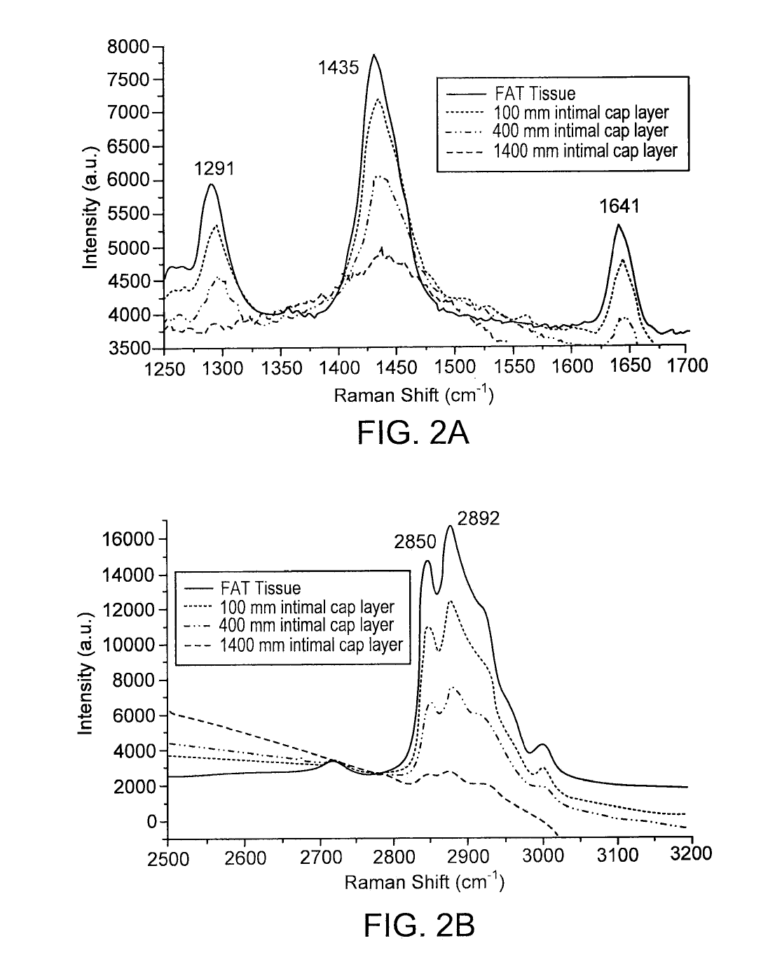

FIG. 2(a) showed Raman spectral intensity changes of Raman mode of 1435 cm⁻¹ in the region 1250 cm⁻¹ to 1700 cm⁻¹ versus the cap thickness of intimal layers on the top of fatty tissue. FIG. 2(b) showed changes at Raman modes of 2850 cm⁻¹ and 2892 cm⁻¹ in the region 2500 cm⁻¹ to 3200 cm⁻¹ for cap thickness. Raman spectral profiles show uniform decreased in intensity of Raman mode vs. cap thickness of tissue overlayers in both scan regions. A fluctuation of Raman spectral intensity occurred at 800 μm of layer thickness for modes of 1435 cm⁻¹, 2850 cm⁻¹ and 2892 cm⁻¹. This error may have been caused by the frozen gel of section layers which will not occur in vivo tissue. Thus, the signal intensity of these Raman bands decreases as the cap tissue layer increases.

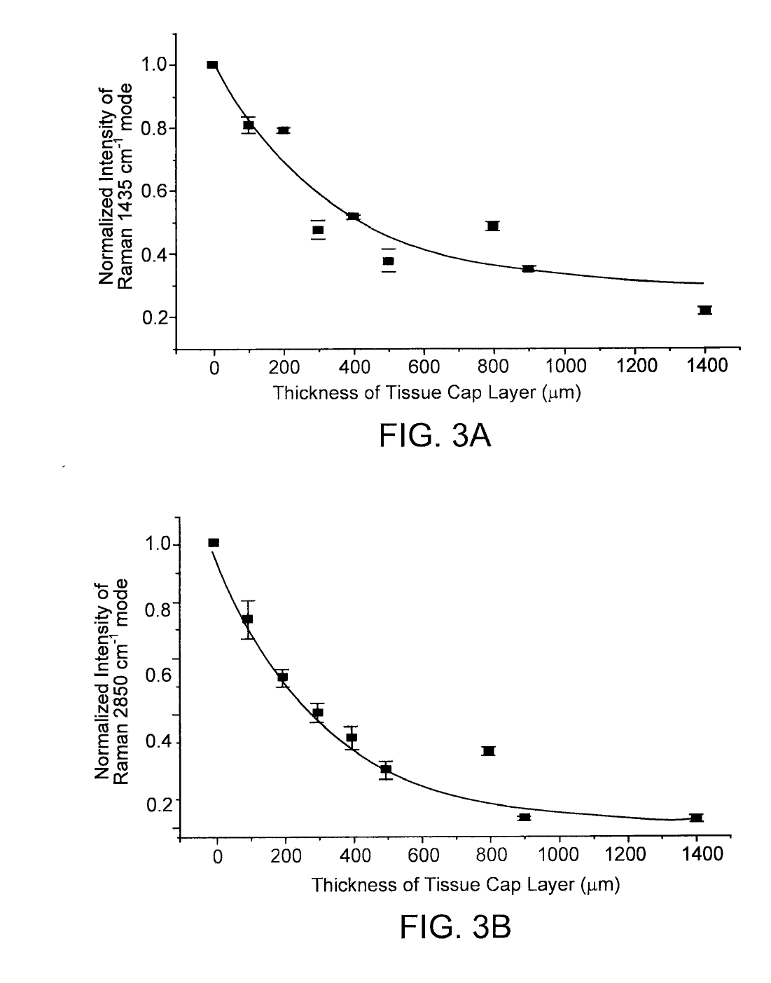

The intensity changes versus the thickness of tissue layers plot in FIGS. 3a and 3b with scan regions set at 300 cm⁻¹ to 1800 cm⁻¹ and 2000 cm⁻¹ to 3200 cm⁻¹, respectively. A first order exponential decay function fits the curves of FIGS. 3a and 3b with the equation: I=I₀e^{-ad}, where d is the thickness of layer in film and a is the attenuation coefficient at 633 nm. An averaged calculation for intensity attenuated to half (I₀/I=0.5), the layer thickness was fit to d=244±128 μm for the 1435 cm⁻¹ mode, with a=0.00284 and d=242±40 μm for the 2850 cm⁻¹ and 2892 cm⁻¹ modes with α=0.00289. This data is in good agreement with in vitro aorta tissue measurements.

The above specification, examples and data provide a description of the apparatus and method of the invention. Since many embodiments of the invention can be made without departing from the spirit and scope of the invention, the invention also resides in the claims hereinafter appended.

1. A method of detecting vulnerable plaque comprising:
a) irradiating a cardiovascular tissue sample with monochromatic light,
b) detecting Raman signal scattering from the cardiovascular tissue sample at one or more of about 1435 cm⁻¹, about 2850 cm⁻¹, or about 2892 cm⁻¹, and at one or more background frequency, and
c) processing the Raman signal to obtain spectroscopic information at one or more of about 1435 cm⁻¹, about 2850 cm⁻¹, or about 2892 cm⁻¹,
wherein the spectroscopic information at one or more of about 1435 cm⁻¹, about 2850 cm⁻¹, or about 2892 cm⁻¹ indicates the presence or absence of vulnerable plaque in the cardiovascular tissue sample.

2. The method of claim 1, wherein said processing step comprises subtracting the Raman signal at a background frequency from the Raman signal at one of about 1435 cm⁻¹, about 2850 cm⁻¹, or about 2892 cm⁻¹ to obtain a background-subtracted peak intensity or area.

3. The method of claim 1, further comprising performing steps a) and b) at multiple locations on said cardiovascular tissue sample and said spectroscopic information is mapped to the multiple locations.

4. The method of claim 1, further comprising the step of locating a fiber optic Raman endoscope near a cardiovascular tissue sample prior to irradiating the cardiovascular tissue sample.

5. The method of claim 1, comprising the step d) determining a thickness of a tissue cap layer by measuring the attenuation of the spectroscopic information at one or more of about 1435 cm⁻¹, about 2850 cm⁻¹, or about 2892 cm⁻¹.

6. The method of claim 5, wherein vulnerable plaque is present if the thickness of the tissue cap is less than 100 μm.

7. The method of claim 1, wherein Raman scatter is detected at both of about 2850 cm⁻¹, and about 2892 cm⁻¹.

8. The method of claim 7, wherein Raman scatter is detected at each of about 1435 cm⁻¹, about 2850 cm⁻¹, and about 2892 cm⁻¹.

9. The method of claim 1, further comprising displaying said spectroscopic information.

10. The method of claim 1, wherein said cardiovascular tissue sample is human arterial tissue.

11. The method of claim 1, wherein said detecting vulnerable plaque is performed in situ in a mammal.

12. The method of claim 1, further comprises detecting calcified plaque regions, wherein step b) comprises detecting Raman scatter at three or more wavenumbers, wherein one of the three or more wavenumbers is about 1435 cm⁻¹, about 2850 cm⁻¹, or about 2892 cm⁻¹,
one of the three or more wavenumbers is about 957 cm⁻¹, and
one of the three or more wavenumbers is a background wavenumber, and
step c) comprises processing the Raman scatter to obtain spectroscopic information at one or more of about 1435 cm⁻¹, about 2850 cm⁻¹, or about 2892 cm⁻¹, and about 957 cm⁻¹,
wherein the spectroscopic information at about 957 cm⁻¹ indicates the presence or absence of calcified plaque in the cardiovascular tissue sample.

13. The method of claim 1, further comprising determining the change of arthrosclerosis lesions, determining the stage of plaque rupture, determining the effect of diet on vulnerable plaque development, or determining the effect of lipid-lowering therapy on vulnerable plaque development.

14. A method of treating a patient comprising, detecting vulnerable plaque as defined in claim 1 and targeting said vulnerable plaque for treatment.

15. An apparatus for detecting vulnerable plaque in a sample comprising:
a source of monochromatic light,
a photodetector,
a probe comprising a fiber optic endoscope, which is adapted to transmit light from the monochromatic light source and light to the photodetector, and
a processor configured to provide spectroscopic information obtained from the photodetector at one or more of about 1435 cm⁻¹, about 2850 cm⁻¹, or about 2892 cm⁻¹, calculate the thickness of sample intimal wall, and indicate the presence or absence of vulnerable plaque in the sample.

16. The apparatus of claim 15, wherein the apparatus is further adapted for detecting calcified plaque regions, said processor being further configured to provide spectroscopic information at about 957 cm⁻¹, and indicate the presence of absence of calcified plaque in the sample.

17. The apparatus of claim 15, further comprising a display adapted for displaying the spectroscopic information and data indicating the presence or absence of vulnerable plaque.

18. The apparatus of claim 15, further comprising one or more narrow band filters selective for Raman scattered light at a Raman shift of one or more of about 1435 cm⁻¹, about 2850 cm⁻¹, or about 2892 cm⁻¹.

19. The apparatus of claim 15, wherein the fiber optic endoscope comprises:
a first channel for transmitting light to the tissue and for collecting an image of the tissue and
a second channel for transmitting monochromatic light to the tissue sample and for collecting Raman scattered light from the sample at a Raman shift of at least one of about 1435 cm⁻¹, about 2850 cm⁻¹, or about 2892 cm⁻¹.
